(19) United States
(12) Patent Application Publication
(10) Pub. No.: US 2005/0025833 A1
(43) Pub. Date: Feb. 3, 2005

(54) Pharmaceutical composition and method for transdermal drug delivery

(76) Inventors: Chaim Aschkenasy, Yerucham (IL); Oren Chen, Beer Sheva (IL); Rami Boochnik, Yerucham (IL); Eilon Asculai, Lehavim (IL); Chalil Abu-Gnim, Beer Sheva (IL); Amira Zeevi, Omer (IL)

Correspondence Address: MARTIN MOYNIHAN;c/o ANTHONY CASTORINA, SUITE 207, 2001 JEFFERSON DAVIS HIGHWAY, ARLINGTON, VA, 22202, US

(21) Appl. No.: 10/891,486

(22) Filed: Jul. 15, 2004

Related U.S. Application Data
(60) Provisional application No. 60/581,458, filed on Jun. 22, 2004.
(60) Provisional application No. 60/487,278, filed on Jul. 16, 2003.
(60) Provisional application No. 60/487,277, filed on Jul. 16, 2003.
(60) Provisional application No. 60/487,248, filed on Jul. 16, 2003.

(51) Int. Cl. A61K 9/14

(52) U.S. Cl. 424/484

(57) ABSTRACT

A pharmaceutical composition for transdermal administration of a hormone (e.g., testosterone), which includes a quaternary ammonium compound as a penetration enhancer, and methods utilizing same for treating medical conditions in which elevating a hormone serum level is beneficial are disclosed.

This application claims the benefit of priority from U.S. Provisional Patent Applications Nos. 60/487,278, 60/487,248, and 60/487,277, filed Jul. 16, 2003, and U.S. Provisional Patent Application No. 60/581,458, filed Jun. 22, 2004, all of which are incorporated by reference as if fully set forth herein,

### FIELD AND BACKGROUND OF THE INVENTION

The present invention relates to novel compositions for the transdermal administration of testosterone and/or other hormones and to methods utilizing these compositions.

Drugs are ideally administered in such a way as to enable an optimal concentration of active agent to be delivered to the intended target site. Conventional routes of administration include ingestion, injection, inhalation, and topical application.

Oral administration is the most prevalent method of administering pharmacological medicaments. The medicament is generally incorporated into a tablet, capsule, or a liquid base, and then swallowed. The oral administration modality is often preferred because of its convenience. In addition, oral administration is generally non-threatening, painless, and simple to accomplish for most patients.

Nevertheless, oral administration of drugs suffers from several disadvantages. One disadvantage is that pediatric and geriatric patients frequently have difficulty swallowing pills and other solid dosage forms, and such patients often refuse to cooperate in swallowing a liquid medication. In addition, for many medicaments, the act of swallowing the medicament often requires fluids and increases gastric volume and the likelihood of nausea and vomiting.

Furthermore, drugs with short half-lives require repeated daily dosing (2 to 4 times daily), which can lead to inadequate compliance. The short plasma half life of the drug and frequent dosing regimen may result in “peaks” and “valleys” in the plasma concentration profile, which increases the likelihood of adverse side effects associated with the peak concentration, as well as decreased therapeutic effectiveness towards the end of the dosing interval.

A further problem associated with oral administration is that the rate of absorption of the drug into the bloodstream after swallowing varies from patient to patient. The absorption of the drug is dependent upon the movement of the drug from the stomach to the small and large intestines and the effects of secretions from these organs and on the resulting pH within the stomach and intestines. Anxiety and stress can dramatically reduce these movements and secretions, prevent or reduce the final effects of the drug, and delay onset of the drug's effects.

An additional disadvantage associated with oral administration is the fact that there is normally a substantial delay between the time of oral administration and the time that the therapeutic effect of the drug begins. As mentioned above, the drug must pass through the gastrointestinal system in order to enter the bloodstream, which typically takes forty-five minutes or longer.

An additional disadvantage of oral administration is that many drugs almost immediately experience metabolism or inactivation. The veins from the stomach and the small and large intestines pass directly through the liver. Thus, drugs entering the bloodstream must first pass through the liver before distribution into the general blood circulation. More than sixty percent of most drugs (and essentially one hundred percent of certain drugs) are removed from the patient's bloodstream during this “first pass” through the liver, resulting in poor bioavailability.

Furthermore, additional stress is placed on the liver as it removes the excess drug from the bloodstream. This is particularly severe if the drug treatment has been occurring over an extended period of time. The liver may become overloaded with the drug's metabolite, which must then be excreted. As a result, there is an increased risk of hepatic or renal disorders.

Transdermal delivery of drugs provides many advantages over conventional oral administration. Advantages of transdermal systems include bypassing the portal circulation, thereby eliminating first-pass metabolism in the liver, convenience, non-interrupted therapy, improved patient compliance, reversibility of treatment (by removal of the transdermal system from the skin), and delivery of medication directly into the system circulation at a constant rate.

Although transdermal systems have many advantages over oral administration, most drugs are not amenable to this mode of administration due to the well-known barrier properties of the skin. Skin is a structurally complex, relatively thick membrane. Molecules moving from the environment into and through intact skin must first penetrate the stratum corneum and any material on its surface. The molecule must then penetrate the viable epidermis, the papillary dermis, and then the capillary walls into the blood stream or lymph channels.

The stratum corneum, the outer horny layer of the skin, is a complex structure composed of dead, keratinized, metabolically inactive cells, which are closely packed together, and consist of an amorphous matrix of mainly lipoid and non-fibrous protein within which keratin filaments are distributed. The cells of the stratum corneum generally contain 20% water, while the cells of the stratum germinativum, situated below the stratum corneum, contain 70% water. The high degree of keratinization within these cells, as well as their dense packing, creates a substantially impermeable barrier to drug penetration, presenting a rate-limiting barrier to absorption of topical compositions or transdermally administered drugs.

In order to improve the penetration of drugs into the skin, a variety of techniques and materials have been developed. These include iontophoresis and ultrasound to improve penetration of drugs into the skin, and the use of formulations containing penetration enhancing compounds, surfactants, lipids and other aliphatic compounds and liposomes.

Penetration enhancers are materials that have a direct effect on the permeability of the skin barrier. Chemical penetration enhancers are believed to operate mainly in the intercellular spaces of the stratum corneum. The exact mechanisms by which many chemical penetration enhancers function have not been clearly elucidated; it is almost certain that they, will have multiple effects once absorbed into the stratum corneum. Effect, that have been documented include an alteration of the solvent potential of the stratum corneum's biochemical environment, and a disordering of the intercellular lipid matrix following insertion of the enhancer into the bilayer structure [“Percutaneous Penetration Enhancers”, E. W. Smith and H. I. Maibach, Eds., CRC Press, 1995].

Modern investigative techniques have shown that many enhancers may operate via a disruption of the ordered structure of the intercellular lipid regions of the stratum corneum. The insertion of the enhancer molecule between the parallel carbon chains of the fatty acids is believed to enhance the fluidity of this environment, thereby facilitating the diffusion of the co-administered drug [“Percutaneous Penetration Enhancers”, E. W. Smith and H. I. Maibach, Eds., CRC Press, 1995].

One method for transdermal delivery involves the use of a patch, which relies on diffusion of the drug through a membrane. A number of transdermal patch delivery systems are known, all of which include at least one adhesive layer for attaching the patch to the target site. These transdermal patch delivery systems suffer some disadvantages, attributed, for example, to the skin irritation caused by the adhesive layer and to their incompatibility for patients having excessively oily or tender skin, or hairy skin.

Topical formulations have also been developed for transdermal delivery, which are applied directly to the skin and release the active ingredient at a rate that is dependent upon the thermodynamic activity of the drug in the formulation. Topical formulations may be applied in the form of, for example, a gel, a cream, an ointment, a paste, a lotion, a milk, a suspension, an aerosol, a spray, a foam, a serum, a swab, a pledget, a pad or a patch.

A topical formulation should be easy to apply, without being too runny, greasy, or otherwise inconvenient to use by the patient. Hydrogels are macromolecular networks that absorb water and swell, but do not dissolve in water, due to the presence of both hydrophilic functional groups that provide for water absorption, and crosslinked polymers that give rise to aqueous insolubility. Hydroalcoholic gels are hydrogels having a high alcohol concentration. These gels have the advantage of ease of administration. At the application site, the alcohol evaporates and it is believed that the drug becomes supersaturated. The skin functions as a reservoir for the drug, which is delivered to the systemic blood circulation at a relatively constant rate and during a period lasting several hours.

Testosterone is the primary endogenous male steroid hormone, produced primarily by the Leydig's cells in the testes in varying amounts throughout a person's lifespan.

The effects of this hormone become most evident during the time of puberty, when an increased output of testosterone will elicit dramatic physiological changes in the male body. This includes the onset of secondary male characteristics such as a deepened voice, body and facial hair growth, increased oil output by the sebaceous glands, development of sexual organs, maturation of sperm and an increased libido. Indeed the male reproductive system will not function properly if testosterone levels are not significant. All such effects are considered the masculinizing or “androgenic” properties of this hormone. Increased testosterone production will also cause growth promoting or “anabolic” changes in the body, including an enhanced rate of protein synthesis (leading to muscle accumulation).

Testosterone also has a number of secondary effects, which are of great importance for the stressability and performance characteristics of the human organism. These include the maintaining of an anabolic metabolic situation, the restoration of the performance of man following exhausting exercise and increasing the psychophysiological stressability and stress resistance.

Over 90% of the testosterone in the blood is bound to protein and the biologically active component is free testosterone representing 4 to 8% of the total concentration in the blood. The testosterone concentration in the blood is subject to a physiological daily cycle (maximum concentration in the morning) a seasonal cycle (lowest concentration in May) and influences by living circumstances and ageing processes.

Testosterone pharmacological uses include hormone replacement therapy in males with a congenital or acquired deficiency or absence of endogenous testosterone (resulting in e.g. hypogonadism, erectile dysfunction), and treatment of AIDS wasting syndrome in HIV infected men.

Hypogonadism may be classified into one of three types. Primary hypogonadism includes testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica). Patients with primary hypogonadism show an intact feedback mechanism in that the low serum testosterone concentrations are associated with high follicle-stimulating hormone (FSH) and leutenizing hormone (LH) concentrations. However, because of testicular or other failures, the high LH concentrations are not effective at stimulating testosterone production.

Secondary hypogonadism involves an idiopathic gonadotropin or LH-releasing hormone deficiency. This type of hypogonadism includes Kaliman's Syndrome, Prader-Labhart-Willi's Syndrome, Laurence-Moon-Biedl's Syndrome, pituitary insufficiency/adenomas, Pasqualim's Syndrome, hemochromatosis, hyperprolactinemia, or pituitary-hypothalamic injury from tumors, trauma, radiation, or obesity. Because patients with secondary hypogonadism do not demonstrate an intact feedback pathway, the lower testosterone concentrations are not associated with increased LH or FSH levels. Thus, these men have low testosterone serum levels but have gonadotropins in the normal to low range.

Third, hypogonadism may be age-related. Testosterone deficiencies in older men may lead to a variety of physiological changes, including sexual dysfunction, decreased libido, loss of muscle mass, decreased bone density, depressed mood, and decreased cognitive function.

Symptoms of low testosterone include decreased sexual desire and ability (decreased libido), extreme tiredness, low energy, depression, and loss of certain male characteristics such as muscular build and deep voice.

The major physiological effects of androgens in normal women include, for example, anabolic effects on muscle, skin, hair and bone; stimulatory effects on erythropoiesis; modulatory effects on immune function; and psychological effects on mood, well-being and sexual function. In addition, endogenous androgens are important for the development of pubic hair and are thought to modulate the action of estrogens and progestins on a variety of reproductive target tissues. It is also believed that androgens play an important role in modulating the secretory function of the lacrimal gland.

Testosterone treatment is generally used in women to treat breast cancer and postpartum breast pain or engorgement to enhance the sex drive, for relief of menopausal symptoms, restoration of lost energy, and to strengthen bone. Testosterone administration has also been found to be beneficial in young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

Testosterone is not effective when taken orally or by injection, because it is susceptible to relatively rapid breakdown by the liver. Systemic administration of testosterone should therefore preferably be effected transdermally.

While many efforts have been made to develop transdermal systems for the delivery of testosterone, in the form of a patch or a topical formulation applied directly to the skin of the subject, only a few have met with commercial success. These include, for example Androgel® and Testim™.

It is believed, in general, that many attempts to produce topical hormone replacement therapies have been unsuccessful due to the inability to adequately and stably target the systemic blood circulation with therapeutically effective dosages in a reasonable period of time. In addition, effective carrier systems, including, for example, solvents for the hormonal drug of interest and suitable percutaneous penetration enhancers, having the requisite product stability and drug delivery profiles, generally cannot be developed based simply on the knowledge of carrier systems in topical formulations for other specific drugs or even from the carriers for patch systems for the same drug.

The background art discloses various topical transdermal formulations for delivery of hormones and other active compounds, which contain any of a wide range of penetration enhancers. For example, U.S. Pat. No. 5,164,190; U.S. Pat. No. 5,152,997; U.S. Pat. No. 5,028,435; U.S. Pat. No. 5,288,498; EP 596903, U.S. Pat. No. 5,788,984, U.S. Pat. No. 4,704,282, and US 2003/0072792 teach transdermal systems for delivery of steroid hormones, including testosterone, in a patch formulation; U.S. Pat. No. 5,198,223 and U.S. Pat. No. 5,314,694 teach systems for transdermal administration of estrogens and progesterone; U.S. Pat. No. 4,788,062 teaches administration of progesterone and estradiol esters; U.S. Pat. No. 5,518,734 teaches administration of estradiol; U.S. Pat. No. 5,512,292 teaches administration of estrogen and gestodene; U.S. Pat. No. 5,236,906 teaches a system for delivery of adrenocortical hormone and hyaluronic acid; U.S. Pat. No. 4,738,956 teaches delivery of hydrocortisone; U.S. Pat. No. 6,420,394 teaches delivery of non-steroidal anti-inflammatory drugs; U.S. Pat. No. 5,874,074 and U.S. Pat. No. 5,658,559 which teach a lotion comprising a dermatological agent, which may be a steroid; U.S. Pat. No. 5,904,931 teaches delivery of sex hormones; and U.S. Pat. No. 5,219,877 teaches delivery of an imidazole; US 2002/0111487 teaches transdermal administration of an active principle such as a hormone, US 2003/015030 teaches delivery of an alpha reductase inhibitor, optionally together with testosterone; US 2003/109507 teaches delivery of progestin; US 2003/087885 teaches dihydrotesterone; and WO 02/22132 teaches delivery of progesterone.

US 2003/0022875 teaches an oral dosage form containing an androgenic agent, which may be co-administered together with a transdermal formulation including a vasoactive agent and a penetration enhancer.

Transdermal delivery systems in which testosterone is mentioned as one of a number of possible active agents include U.S. Pat. No. 5,733,572 and U.S. Pat. No. 6,313,715, which teaches a delivery system comprising gas filled microspheres, comprising one of a list of possible penetration enhancers; U.S. Pat. No. 5,505,958, U.S. Pat. No. 5,744,162; U.S. Pat. No. 5,891,463, U.S. Pat. No. 5,902,603, and US 2003/0082227, which teach use of various penetration enhancers in a system comprising a patch; U.S. Pat. No. 4,946,870, which teaches a system comprising a film-forming system comprising an aminopolysaccharide, and includes any one of a list of penetration enhancers; U.S. Pat. No. 6,267,984, which uses monoglyceride and ethyl palmitate as penetration enhancers; US 2002/0028235 which uses an ester sunscreen as penetration enhancer, U.S. Pat. No. 4,863,970, which uses a binary system of oleic acid, oleins, and oleyl alcohol with lower alcohols to enhance penetration; US 2002/0058650, which uses a penetration enhancing system comprising oleic acid, alcohol and glycol; US 2002/0150625, which teaches a poloxamer lecithin organogel as transdermal carrier, EP 644746, which teaches ethanol, water, glycerol monoleate and methyl laureate as enhancer; U.S. Pat. No. 6,019,988, which teaches use of separate permeation enhancer and drug compositions which are mixed at time of application; and U.S. Pat. No. 5,023,252 which teaches use of a compound which may be a macrocyclic ester, diester, amide, diamide, amidine, diamidine, thioester, dithioester, thioamide, ketone or lactone.

Use of various other penetration enhancers is taught in the background art. U.S. Pat. No. 4,804,541 teaches use of benzyl alcohol; WO 95/05137 teaches a permeation enhancer composition, which includes benzyl alcohol, propylene glycol monolaurate and a C2-C6 alkanediol; U.S. Pat. No. 5,760,096 teaches use of a glycol and an alcohol; U.S. Pat. No. 5,885,565 teaches use of a sterol; U.S. Pat. No. 6,319,913 teaches oleic acid; U.S. Pat. No. 5,723,114 teaches use of a proton pump inhibitor; U.S. Pat. No. 6,190,894 teaches enhancement of penetration by agents which cause inhibition of biosynthesis of epithelial components; U.S. Pat. No. 6,010,691 teaches stearylamine and transvaccenic acid; and U.S. Pat. No. 4,678,663 teaches a volatile silicone and a fatty alcohol; and U.S. Pat. No. 5,894,019 teaches a system in which the active ingredient is dissolved in a liquid lipid to enhance penetration.

Topical transdermal formulation are also taught in the following scientific publications: Funke A P et al, Pharm Res 19:661-8 (2002); and Coldman M F et al, J Pharm Sci 58:1098-102 (1969).

In recent years, various papers and patents have been published, relating to use of hydroalcoholic gels for the transdermal delivery of hormones such as testosterone or dihydrotestosterone.

However, hydroalcoholic gels provide delivery rates which are generally not sufficiently high; therefore a large amount of gel must be applied to a relatively large skin surface area. Therefore, several publications and patents recommend the addition of certain penetration enhancers to the gel.

Examples of such publications include U.S. Pat. No. 6,503,894, U.S. Pat. No. 6,019,997, WO 02/17926, US 2003/0022877, US 2003/27804, and US 2003/0050292 which describe a hydroalcoholic gel, comprising an androgenic or anabolic steroid, which may be testosterone, and a functional derivative of a fatty acid as penetration enhancer; US 2002/0183296 and WO 02/17927, which teach a hydroalcoholic gel comprising testosterone and a fatty acid derivative penetration enhancer; and US 2003/0087885, which teaches delivery of dihydrotesterone; U.S. Pat. No. 5,968,919 which teaches topical alcoholic or aqueous alcoholic gels containing testosterone, progesterone, or estradiol, with 2-n-nonyl-1,3-dioxolane or other hydrocarbyl derivative of 1,3-dioxolane or 1,3-dioxane or acetal as penetration enhancers; and U.S. Pat. No. 5,908,619 which optionally uses one of a variety of penetration enhancers. Another drawback of the hydroalcoholic gels is the sticky feeling caused by the gelling agents remaining on the skin after the evaporation of the alcohol. The commercial products Androgel® and Testim™ contain isopropyl myristate and pentadecalactone, respectively, as penetration enhancers. With both products a large amount of the product (5-10 grams) must be applied to the shoulders or the abdomen. With both products only a fraction of the applied testosterone reaches the systemic blood circulation.

The prior art therefore does not teach a topical composition for transdermal delivery of testosterone or related hormones, having a particularly effective penetration level and characterized by convenient and effective application.

Formulations having a penetration enhancer achieving desired serum drug levels cannot be developed based simply on the knowledge of carrier systems in topical formulations for other specific drugs. Moreover, even penetration enhancers belonging to the same chemical class (fatty acid and esters, polyols and surfactants among others) have sometimes quite different influences on penetration rates.

There is thus a widely recognized need for, and it would be highly advantageous to have, topical formulations, preferably hydrogel formulations, which include testosterone, and a highly effective penetration enhancer, and can therefore serve for transdermally delivering testosterone while being devoid of at least some of is the above limitations.

### SUMMARY OF THE INVENTION

The present inventors have surprisingly found that hydroalcoholic gels that comprise a quaternary ammonium compound as a penetration enhancer are particularly effective systems for transdermal delivery of testosterone and related hormones.

Hence, according to one aspect of the present invention there is provided a hydroalcoholic gel for topical application, which comprises a pharmaceutically active ingredient, a penetration enhancer, an alcohol and a gelling agent, wherein the pharmaceutically active ingredient is a hormone, and the penetration enhancer is a quaternary ammonium compound.

The quaternary ammonium compound may be selected from the group consisting of hexadecyltrethylammonium bromide, methylbenzethonium chloride, cetalkonium chloride, centrimonium bromide, domiphen bromide, domiphen chloride, domiphen fluoride, cetyl pyridinium chloride, dequalinium chloride, the cis isomer of 1-(3-chloroallyl)3,5,7-triaza-1-azoniaadamantane chloride, cetyl trimethylammonium bromide, benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; N,N-(diethyl-N-[2-[4(1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride.

Preferably, the quaternary ammonium compound is benzalkonium chloride, benzethonium chloride, cetrimide or methylbenzethonium chloride. More preferably, the quarternary ammonium compound is benzalkonium chloride.

The hormone is preferably any one or more of an androgenic hormone, an estrogenic hormone and a progestogenic hormone, and can be, for example, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronodiol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozlol, testosterone, testosterone decanoate, 4-dihydrotestosterone, 5α-dihydrotestosterone, testolactone, 17α-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone, estrone, estradiol and estriol, progesterone and pharmaceutically acceptable esters thereof, salts thereof, and combinations of any of the foregoing.

Preferably, the hormone is testosterone.

The concentration of the hormone preferably ranges between about 0.5 weight percentages and about 5 weight percentages, more preferably it is about 1 weight percentage.

In one embodiment of the present invention, the concentration of quaternary ammonium compound is preferably in the range of from about 0.04 weight percentages to about 0.4 weight percentages, more preferably in the range of from about 0.07 weight percentages to about 0.1 weight percentages.

The hydroalcoholic gel pharmaceutical composition of the present invention preferably comprises a C2-C4 alcohol, such as, for example, ethanol or isopropanol, preferably ethanol. The concentration of the C2-C4 alcohol preferably ranges between about 40 weight percentages and about 90 weight percentages, more preferably between about 55 weight percentages and about 70 weight percentages, and most preferably is about 69 weight percentages.

The hydroalcoholic gel composition preferably further comprises a gelling agent, such as, for example, a polymeric thickening agent, a fatty alcohol, a fatty acid, and a fatty acid alkali salt, an inorganic gelling agent and any mixture thereof, more preferably a polyacrylic acid or a cellulosic ether. Preferred cellulosic ethers are carboxymethylcellulose, hydroxypropyl cellulose and hydroxyethylcellulose. Preferred polymeric thickening agent are xanthan gum and guar gum

The concentration of the gelling agent preferably ranges between about 0.1 weight percentage and about 5 weight percentages, more preferably between about 0.1 weight percentage and about 2 weight percentages.

The pharmaceutical composition according to the present invention can further comprise a penetration co-enhancer, optionally and preferably a glycol.

The composition may optionally further comprise an additional pharmaceutically active ingredient.

The pharmaceutical composition optionally further comprises at least one additive, optionally and preferably selected from the group consisting of a moisturizing agent and an emollient. The additive optionally comprises glycerin. The additive may alternatively be an emollient selected from the group comprising dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof. Alternatively, the additive may be selected from the group consisting of a humectant, a deodorant agent, an antiperspirant, a pH adjusting agent, a stabilizing agent, a preservative, an emulsifier, an occlusive agent, a solubilizing agent, a colorant, and a surfactant. The concentration of additive preferably ranges between about 1 weight percentage and about 5 weight percentages.

In a preferred embodiment of the present invention, the hydroalcoholic gel composition, as described hereinabove, comprises benzalkonium chloride, testosterone, a C2-C4 alcohol and a gelling agent.

The pharmaceutical composition of the present invention is capable, upon application of an amount of the composition onto at least one biological surface of a subject, of elevating a blood serum concentration of the hormone in the subject from a subpotent concentration to a potent concentration within about 24 hours after application. For testosterone, in a human male, a potent concentration ranges from about 300 ng/dl to 1100 ng/dl in serum.

The amount of pharmaceutical composition preferably ranges between about 0.1 grams and about 10 grams. Alternatively, the amount of the composition preferably ranges between about 3 milligrams and 100 milligrams, more preferably between about 4 milligrams and about 60 milligrams per square centimeter of the biological surface. In the case where the hormone is testosterone, the amount of composition may be lower than these values.

The biological surface can be, for example, the abdomen, an armpit, an inside arm, the back, a thigh, a shoulder, or the scrotum.

The pharmaceutical composition can therefore be packaged in a packaging material and identified in print, in or on the packaging material, for use in the treatment of a medical condition in which elevating a blood serum hormone level in a subject is beneficial.

In cases where the subject is a human male, the medical condition can be, for example, primary hypogonadism, secondary hypogonadism, age-related hypogonadism, hormone deficiency, erectile dysfunction, AIDS wasting syndrome, reduced sex drive, energy loss, loss of bone mass, extreme tiredness, low energy, and/or depression.

In cases where the subject is a human female, the medical condition can be, for example, breast cancer, postpartum breast pain or engorgement, reduced sex drive, menopausal symptoms, energy loss, loss of bone mass, extreme tiredness, low energy, and/or depression. The human female may be, for example, young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

According to another aspect of the present invention there is provided a method of transdermally delivering a hormone to the blood serum of a subject. This method comprises providing a hydroalcoholic gel pharmaceutical composition for topical application which includes the hormone, a quaternary ammonium compound, an alcohol, a gelling agent; and contacting an amount (e.g, about 5 grams for a composition comprising testosterone) of the topical pharmaceutical composition with at least one biological surface of the subject, to thereby deliver the hormone to the blood serum through the biological surface.

According to still another aspect of the present invention there is provided a method of treating a medical condition in which elevating a hormone serum level is beneficial. The method comprises topically applying onto one or more biological surface(s) of a subject in need thereof, a pharmaceutically effective amount of the hydroalcoholic gel composition described hereinabove.

The hormone blood serum level, the amount of the composition, the subject, the medical condition and the biological surface are as described hereinabove.

An advantage of the compositions and/or the methods of the present invention is that the transdermal penetration of a hormone is substantially increased.

A further advantage of the compositions and/or the methods of the present invention is that a smaller amount of a hormone can be administered, while still achieving a desired serum hormone level.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the phrase “topical application” describes application onto a biological surface. Hence, the phrase “a composition for topical application” describes a composition that is applied to a subject by contacting one or more biological surface(s) of the subject.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “pharmaceutically active ingredient” refers to a pharmaceutical agent including any natural or synthetic chemical substance that subsequent to its application has, at the very least, at least one desired pharmaceutical effect.

The term “penetration enhancement” refers to an increase in the permeability of skin to a pharmacologically active agent, so as to increase the rate at which the drug permeates through the skin and enters the bloodstream. The enhancement can be observed by measuring the rate of diffusion of drug through animal or human skin using, for example a Franz diffusion apparatus as known in the art.

“Carriers” or “vehicles” refer to carrier materials suitable for transdermal drug administration and include any such material known in the art, e.g. any liquid, gel, solvent, liquid diluent, solubilizer or the like, which is nontoxic and which does not interact with other components of the composition in a deleterious manner. Examples of suitable carriers include water, alcohols, mineral oil, silicone, liquid sugars, waxes, petroleum jelly, and a variety of other oils and polymeric materials.

The term “transdermal” is intended to denote both transdermal (or “percutaneous”) and transmucosal administration, i.e., delivery by passage of drug through the skin or mucosal tissue. Hence the terms “transdermal” and “transmucosal” are used interchangeably unless specifically stated otherwise.

The term “therapeutically effective amount” or “pharmaceutically effective amount” denotes that dose of pharmaceutically active ingredient that will provide the pharmacological effect for which the active ingredient is indicated.

By “drug composition”, “drug/enhancer composition” or any similar terminology is meant a formulated composition containing the drug to be transdermally delivered in combination with such “carriers” or “vehicles”, penetration enhancers, excipients, or any other additives.

As used herein, the phrase “pharmaceutically acceptable carrier” describes a carrier that does not cause significant irritation to an organism and does not abrogate the biological activity and properties of the applied active ingredient.

As used herein, the phrase “potent concentration” with regard to a hormone denotes a concentration at which the hormone exerts a physiological effect. The phrase “subpotent concentration” denotes a concentration of the hormone which is below the potent concentration level.

As used herein the term “about” refers to ±10%.

The phrase “weight percentage(s)” or “percent” describes the weight percentage(s) of an ingredient of the total weight of a composition containing the ingredient.

The phrase “greater than” as used herein with respect to a numerical indication (e.g., a concentration) encompasses any number (integral or fractional) that is greater than the indicated number.

The phrase “lower than” as used herein with respect to a numerical indication (e.g., a concentration) encompasses any number (integral or fractional) that is lower than the indicated number.

The phrase “ranging between” or “ranges between” as used herein with respect to a first numerical indication and a second numerical indication, and the phrase “ranging from” or “ranges from” with respect to a first numerical indication and “to” with respect to a second numerical indication, are used interchangeably, and are meant to include the first and second numerical indications, as well as all integral and fractional numerals therebetween.

### DESCRIPTION OF THE PREFERRED EMBODIMENTS

The present invention is of a novel hydroalcoholic gel composition for transdermal delivery of a hormone (e.g., testosterone), using a quaternary ammonium compound as penetration enhancer, which can be beneficially used in the treatment of medical conditions in which elevating the hormone serum level in a subject is beneficial, such as, but not limited to, hypogonadism, erectile dysfunction, hormone deficiency, depression, AIDS wasting syndrome, breast cancer, postpartum breast pain or engorgement, reduced sex drive, menopausal symptoms, energy loss, and loss of bone mass.

The principles and operation of the compositions, processes and methods according to the present invention may be better understood with reference to the Examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The prior art teaches various formulations for transdermally delivering testosterone or other hormones. Most of these formulations are in the form of a patch, and therefore suffer major limitations, as is discussed in detail hereinabove. Alternative forms, such as, for example, emulsions, creams, aqueous solutions, oils, ointments, and pastes generally have the disadvantages of being runny, greasy, or otherwise inconvenient to use by the patient.

While conceiving the present invention, it was envisioned that quaternary ammonium can be efficiently used to enhance the skin permeation of testosterone and other hormones, and thus, that a hydroalcoholic gel composition for topical application that comprises a hormone and a quaternary ammonium compound can be used for efficient transdermal delivery of the hormone. The advantages of hydroalcoholic gels for transdermally delivering drugs are set forth hereinabove.

A “quaternary ammonium compound” refers to a tetravalent nitrogen-containing molecule having a positive charge on the nitrogen atom and a counter ion. Quaternary ammonium compounds oftentimes include aliphatic and aromatic substituents on the nitrogen atom, which stabilize the positive charge. Examples of aliphatic quaternary ammonium compounds include tetraalkyl ammonium chlorides, such as tetramethyl ammonium chloride, tetraethyl ammonium chloride, etc. Examples of aromatic quaternary ammonium compounds include benzalkonium chloride, benzethonium chloride and methylbenzethonium chloride.

Various prior art documents teach benzalkonium chloride as one of a list of possible penetration enhancers. For example, U.S. Pat. No. 6,562,369 and US 2003/0129220 teach use of benzalkonium chloride as a possible co-enhancer, together with a hydroxide-releasing agent, in a system which may be used for delivery of an androgenic hormone; US 2002/0099003 teaches use of benzalkonium chloride as a possible penetration enhancer for delivery of a vasoactive agent; and US 2003/0104041 teaches use of benzalkonium chloride as one of many possible penetration enhancers for delivery of an active compound which may be testosterone. None of these documents teach benzalkonium chloride or other quaternary ammonium compounds as particularly effective penetration enhancers for delivery of hormones such as testosterone.

US 2002/0014307 teaches use of a patch to deliver a vaporizable medicine, which may be a hormone, with a penetration enhancer which may be selected from any one of a large group, including benzalkonium chloride and benzethonium chloride. This document does not teach these quaternary ammonium compounds as being preferred penetration enhancers. Furthermore, the concentration of penetration enhancer used is taught as being in the range of from 0.1 weight percentage to 5.0 weight percentages. This document teaches away from the use of a concentration of penetration enhancer of less than 0.1 weight percentages, stating that if less than 0.1% by weight is used, a sufficient absorption promoting effect cannot be obtained.

US 2003/0091620 teaches transdermal drug delivery systems using quaternary ammonium salts as penetration enhancers, which may be used for delivery of hormones, including testosterone. The penetration enhancer is used at a concentration in the range of from about 0.1% to about 4.5% by weight of the carrier. This document compares the effects of benzethonium chloride in increasing testosterone flux from a pressure sensitive adhesive matrix patch with the effects in increasing testosterone flux from a hydroalcoholic gel. Results obtained indicated that a 1% concentration of benzethonium chloride in a hydroalcoholic gel formulation is insufficient as a penetration enhancer for testosterone. This document therefore teaches away from the use of a quaternary ammonium compound at a concentration of 1% or less, in a hydroalcoholic gel formulation for transdermal delivery of testosterone.

Hence, the prior art does not teach or suggest a hydroalcoholic gel composition comprising testosterone or other hormone as the main active ingredient, with a quaternary ammonium compound as an efficient penetration enhancer.

While reducing the present invention to practice, it was surprisingly found that using a quaternary ammonium compound as a penetration enhancer for increasing the skin permeability of testosterone, results in substantially enhanced testosterone permeability. As is shown in the Examples section that follows, such an enhanced skin permeability enables the use of a minimal amount of the applied composition and a minimal skin surface area onto which the composition is applied, while still achieving a desired testosterone level in a receiving medium (e.g., serum).

The amount of the pharmaceutical composition of the present invention which is required to produce the desired effect therefore preferably ranges between about 0.1 grams and about 10 grams. Alternatively, the amount of the composition ranges between about 3 milligrams and 100 milligrams, preferably between about 4 milligrams and about 60 milligrams per square centimeter of a biological surface onto which it is applied.

Hence, according to one aspect of the present invention, there is provided a hydroalcoholic gel composition which comprises a hormone, as a pharmaceutical active ingredient and a quaternary ammonium compound as a penetration enhancer, aimed at enhancing the skin penetrating effect of the active ingredient.

According to the present invention, the pharmaceutically active ingredient is a hormone. Suitable hormones for use in the context of the present invention include, for example, androgenic compounds, progestogenic compounds, including progestin compounds and progesterone, estrogenic compounds, and a combination thereof.

Representative examples of androgenic compounds include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, pharmaceutically acceptable esters thereof, salts thereof, and combinations of any of the foregoing.

Representative examples of progestogenic compounds include, without limitation, progesterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone and combinations of any of the foregoing.

Representative examples of estrogens include, without limitation, estrone, estradiol, estriol and derivatives thereof.

In a preferred embodiment of the present invention, the hormone is an androgenic hormone, and, more preferably, it is testosterone.

The hormone concentration preferably ranges between about 0.5 weight percentage and about 5 weight percentages, more preferably between about 0.5 weight percentage and about 4 weight percentages, more preferably between about 0.5 weight percentage and about 3 weight percentages, more preferably between about 0.5 weight percentage and about 2 weight percentages, with a presently most preferred concentration being about 1 weight percentage.

As is shown in the Examples section that follows, it was surprisingly found that benzalkonium chloride is an extremely effective penetration enhancer for delivery of a hormone. The concentration of benzalkonium chloride needed to achieve an enhanced skin permeability of a hormone and as a result, an elevated level of the hormone in a receiving medium, is relatively low.

While benzalkonium chloride is the most preferred penetration enhancer according to the present invention, other quaternary ammonium compounds can also be beneficially used as penetration enhancers in the composition of the present invention, in addition to or instead of benzalkonium chloride. Such quaternary ammonium compounds include, for example, benzethonium chloride, methylbenzethonium chloride, hexadecyltrethylammonium bromide, methylbenzethonium chloride, cetalkonium chloride, centrimonium bromide, domiphen bromide, domiphen chloride, domiphen fluoride, cetyl pyridinium chloride, dequalinium chloride, the cis isomer of 1-(3-chloroallyl)-3,5,7-triaza-1-azoniaadamantane chloride, cetyl trimethylammonium bromide, benzalkonium saccharinate; behenalkonium chloride; cetalkonium chloride; erucalkonium chloride; lauralkonium chloride; myrstalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quatenrium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; N,N-(diethyl-N-[2-[4-(1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride.

The concentration of the quaternary ammonium compound is preferably equal to or lower than 0.4 weight percentages and can therefore be, for example, 0.4 weight percentages, 0.35 weight percentages, 0.3 weight percentages, 0.25 weight percentages, 0.2 weight percentages, 0.15 weight percentages, 0.1 weight percentages, 0.09 weight percentages, 0.08 weight percentages, 0.07 weight percentages, 0.06 weight percentages, 0.05 weight percentages and down to 0.01 weight percentages of the total weight of the composition. The concentration of the quaternary ammonium compound therefore preferably ranges from about 0.04 weight percentages to about 0.4 weight percentages, more preferably from about 0.04 weight percentages to about 0.3 weight percentages, more preferably from about 0.04 weight percentages to about 0.2 weight percentages, more preferably from about 0.04 weight percentages to about 0.1 weight percentages, and more preferably about 0.07 weight percentages to about 0.1 weight percentages of the total weight of the composition. The concentration of the quaternary ammonium compound can further preferably range from about 0.04 weight percentage to about 0.09 weight percentage.

As is well known in the art, it is possible to enhance the effect of penetration enhancers by the co-inclusion of additional carriers or enhancers, such as glycols. Irritation caused by penetration enhancers or other ingredients may be reduced by using a combination of enhancers, thereby reducing the amount of each individual enhancer compound, or by the inclusion of non-irritating ingredients such as glycerin.

Hence, according to an embodiment of the present invention, the composition ether comprises a penetration co-enhancer such as glycol or glycerin. Other suitable penetration co-enhancers for use in the context of the present invention include, without limitation, acetone, acyl lactylates, acyl peptides, acylsarcosinates, alkanolamine salts of fatty acids, alkyl benzene sulphonates, alkyl ether sulphates, alkyl sulphates, anionic surface-active agents, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyglycerol esters, polyoxyethylene lauryl ether, polyoxy:polyoxyethylene stearate, polyoxypropylene 15 stearyl ether, poly(vinyl pyridinium chloride), propan-1-ol, propan-2-ol, propylene glycol dipelargonate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium, lauryl ether sulphate, sodium lauryl sulphate, sugar esters, sulphosuccinate, tetrahydrofuran, tetrahydrofurfural alcohol, transcutol, triethanolamine dodecyl benzene sulphonate, triethanolamine oleate, water and derivatives, esters, salts and mixtures thereof.

The amount of co-enhancer used is selected so as to provide the desired delivery rate for the active ingredient, so as to enable application of a minimal amount of the composition onto a minimal skin surface area, taking into account the effect of additional factors such as product stability, side-effects, carrier system, and the like.

Further according to the present invention, the composition further comprises a pharmaceutically acceptable carrier suitable for hydroalcoholic gel formulations Such a pharmaceutically acceptable carrier typically comprises an alcohol and a gelling agent.

Preferably, the alcohol is a C2-C4 alcohol such as, for example, ethanol and/or isopropanol. The concentration of the alcohol preferably ranges between about 40 weight percentages and about 90 weight percentages, more preferably between about 50 weight percentages and about 80 weight percentages more preferably between about 55 weight percentages and about 75 weight percentages, more preferably between about 55 weight percentages and about 70 weight percentages and most preferably between about 65 weight percentages and about 70 weight percentages.

The gelling agent is preferably a thickening agent, such as polyacrylic acid. However, any other pharmaceutically acceptable thickening/gelling agent may be used, such as hydroxypropyl cellulose, hydroxyethylcellulose, or other cellulosic ethers, other polymeric thickening agents such as xanthan gum, guar gum, and the like, fatty alcohols, fatty acids and their alkali salts and mixtures thereof, as well as inorganic thickeners/gelling agents.

The amount of gelling agent is not particularly critical, and can be selected to provide the desired product consistency or viscosity to allow for easy application to the skin. Generally, depending upon its molecular weight, amounts of thickening agent of up to about 5 weight percentages, preferably from about 0.1 weight percentages to about 2 weight percentages, of the composition will provide the desired effect.

The composition of the present invention may further comprise one or more inactive ingredients, which provide the compositions with additional usage benefits. Such inactive ingredients are referred to herein as “additives”. Examples of such additives include, but are not limited to, humectants, deodorant agents, antiperspirants, stabilizing agents, pH adjusting agents, preservatives, emulsifiers, occlusive agents, solubilizing agents, colorants, and surfactants.

Representative examples of pH adjusting agents that are usable in the context of the present invention include, without limitation, a base, an acid, a buffer system, or any combination thereof. Examples of such pH adjusting agents include, but are not limited to, bases such as ammonium hydroxide, sodium hydroxide, calcium hydroxide, trolamine, diisopropanolamine, and triisopropanolamine.

Representative examples of deodorant agents that are usable in the context of the present invention include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmithyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides suh as L-lysine hexadecyl amide, heavy metal salts of citrate, salicylate, and piroctose, especially zinc salts, and acids thereof, heavy metal salts of pyrithione, especially zinc pyrithione and zinc phenolsulfate. Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the compositions of the present invention either in a solubilized or a particulate form and include, for example, aluminum or zirconium astringent salts or complexes.

Suitable preservatives that can be used in the context of the present composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof.

Suitable emulsifiers that can be used in the context of the present invention include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents that can be used in the context of the present invention include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Representative examples of solubilizing agents that are usable in this context of the present invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as tweens and spans e.g., TWEEN 80. Other solubilizers that are usable for the compositions of the present invention are, for example, polyoxyethylene sorbitan fatty acid ester, polyoxyethylene n-alkyl ethers, n-alkyl amine n-oxides, poloxamers, organic solvents, phospholipids and cyclodextrines.

In another preferred embodiment of the present invention, the composition includes at least one additional pharmaceutically active ingredient in addition to the hormone described above.

Suitable additional pharmaceutically active ingredients include but are not limited to active herbal extracts, acaricides, age spot and keratose removing agents, analgesics, local anesthetics, antiacne agents, antiaging agents, antibacterials, antibiotics, antiburn agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, 5-α reductase inhibitors, sanatives, scabicides, vasodilators and wart removers.

Suitable active herbal extracts include but are not limited to angelica, anise oil, astragali radix, azalea, benzyl acetate, birch tar oil, bornyl acetate, cacumen biotae, camphor, cantharidin, capsicum, cineole, cinnamon bark, cinnamon leaf, citronella, citronellol, citronellyl acetate, citronellyl formate, eucalyptus, eugenyl acetate, flos carthami, fructus mori, garlic, geraniol, geranium, geranyl acetate, habanera, isobutyl angelicate, lavender, ledum latifolium, ledum palustre, lemongrass, limonene, linalool, linalyl acetate, methyl anthranilate, methyl cinnamate, mezereum, neem, nerol, neryl acetate, nettle root extract, oleum ricini, oregano, pinenes, α-pinene, β-pinene, radix angelicae sinesis, radix paenoiae rubra, radix polygoni multiflori, radix rehmanniae, rhizoma pinelliae, rhizoma zingiberis recens, sabadilla, sage, sandalwood oil, saw palmetto extract, semen sesami nigrum, staphysagria, tea tree oil, terpene alcohols, terpene hydrocarbons, terpene esters, terpinene, terpineol, terpinyl acetate and derivatives, esters, salts and mixtures thereof.

Suitable acaricides include but are not limited to amitraz, flumethrin, fluvalinate and derivatives, esters, salts and mixtures thereof.

Suitable age spot and keratoses removing agent include but are not limited to hydroxyacids, hydroquinone and derivatives, esters, salts and mixtures thereof.

Suitable analgesics include but are not limited to benzocaine, butamben picrate, dibucaine, dimethisoquin, dyclonine, lidocaine, pramoxine, tetracaine, salicylates and derivatives, esters, salts and mixtures thereof.

Suitable local anesthetics include but are not limited to benzocaine, bupivacaine, butamben picrate, chlorprocaine, cocaine, dibucaine, dimethisoquin, dyclonine, etidocaine, hexylcaine, ketamine, lidocaine, mepivacaine, pramoxine, procaine, tetracaine, salicylates and derivatives, esters, salts and mixtures thereof.

Suitable antiacne agents include but are not limited to N-acetylcysteine, adapalene, azelaic acid,-benzoyl peroxide, cholate, clindamycin, deoxycholate, erythromycin, flavinoids, glycolic acid, meclocycline, metronidazole, mupirocin, octopirox, phenoxy ethanol, phenoxy proponol, pyruvic acid, resorcinol, retinoic acid, salicylic acid, scymnol sulfate, sulfacetamide-sulfur, sulfur, tazarotene, tetracycline, tretinoin triclosan and derivatives, esters, salts and mixtures thereof.

Suitable antiaging agents include but are not limited to melatonin and derivatives, esters, salts and mixtures thereof.

Suitable antibiotics include but are not limited to amanfadine hydrochloride, amanfadine sulfate, amikacin, amikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocylcline, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, metronidazole, miconazole, miconazole hydrochloride, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, yrothricin and derivatives, esters, salts and mixtures thereof.

Suitable antidepressants include but are not limited to norepinephrine-reuptake inhibitors, selective-serotonin-reuptake inhibitors, monoamine-oxidase inhibitors, serotonin-and-noradrenaline-reuptake inhibitors, corticotropin-releasing factor antagonists, αα-adrenoreceptor antagonists, NK1-receptor antagonists, 5-HT_{1A}-receptor agonist antagonists, amitriptyline, desmethylamitriptyline, clomipramine, doxepin, imipramine, imipramin-oxide, trimipramine, adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, tianeptine, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritanserin, sertraline, tandospirone, venlafaxine and zimeldine and derivatives, esters, salts and mixtures thereof.

Suitable antihistamines include but are not limited to chlorcyclizine, diphenhydramine, mepyramine, methapyrilene, tripelennamine and derivatives, esters, salts and mixtures thereof.

Suitable antimycotics include but are not limited to azole compounds, butoconazole, chloroxine, ciclopirox olamine, clotrimazole, econazole, elubiol, fluconazole, griseofulvin, itraconazole, ketoconazole, mafenide acetate, miconazole, nystatin, oxiconazole, sulconazole, terbinafine, terconazole, tioconazole, undecylenic acid and derivatives, esters, salts and mixtures thereof.

Suitable antipruritics include but are not limited to menthol, methdilazine, trimeprazine, urea and derivatives, esters, salts and mixtures thereof.

Suitable antipsoriatic agents include but are not limited to 6-aminonicotinamide, 6-aminonicotinic acid, 2-aminopyrazinamide, anthralin, calcipotriene, 6-carbamoylnicotinamide, 6-chloronicotinamide, 2-carbamoylpyrazinamide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, trichothecene mycotoxins and derivatives, esters, salts and mixtures thereof.

Suitable additional antirosacea agents include but are not limited to metronidazole, sulfacetamide and derivatives, esters, salts and mixtures thereof.

Suitable antiseborrheic agents include but are not limited to glycolic acid, salicylic acid, selenium sulfide, zinc pyrithione and derivatives, esters, salts and mixtures thereof.

Suitable antiviral agents include but are not limited to acyclovir and derivatives, esters, salts and mixtures thereof.

Suitable chemotherapeutic agents include but are not limited to daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.

Suitable corticosteroids include but are not limited to alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, diflurosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, α-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters, salts and mixtures thereof.

Suitable keratolytic agents include but are not limited to N-acetylcysteine, glycolic acid, pyruvic acid, resorcinol, sulfur, salicyclic acid, retinoic acids and derivatives, esters, salts and mixtures thereof.

Suitable lactams include but are not limited to L-galactono-1,4-lactam, L-arabino-1,5-lactam, D-fucono-1,5-lactam, D-glucaro-1,4-lactam, D-glucurono-6,3-lactam, 2,5-tri-O-acetyl-D-glucurono-6,3-lactam, 2-acetamido-2-deoxyglucono-1,5-lactam, 2-acetamido-2-deoxygalactono-1,5-lactam, D-glucaro-1,4:6,3-dilactam, L-idaro-1,5-lactam, 2,3,5,tri-O-acetyl-D-glucaro-1,4lactam, 2,5-di-O-acetyl-D-glucaro-1,4:6,3-dilactam, D-glucaro-1,5-lactam methyl ester, 2-propionoamide-2-deoxyglucaro-1,5-lactam and derivatives, esters, salts and mixtures thereof.

Suitable non-steroidal anti-inflammatory agent include but are not limited to oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof.

Suitable pediculicides include but are not limited to DDT, lindane, malathion, permethrin and derivatives, esters, salts and mixtures thereof.

Suitable vasodilators include but are not limited to ethyl nicotinate, capsicum extract and derivatives, esters, salts and mixtures thereof.

Suitable wart removers include but are not limited to imiquimod, podophyllotoxin and derivatives, esters, salts and mixtures thereof.

The compositions of the present invention may be packed or presented in any convenient way. For example, they may be packed in a tube, a bottle, a unit dosage form or a pressurized container, using techniques well known to those skilled in the art and as set forth in reference works such as Remington's Pharmaceutical Science 15^{th} Ed. Optionally and preferably, the composition is packed in the form of a tube or a unit dosage form, such as a sachet. It is preferred that the packaging is done in such a way so as to minimize contact of the unused compositions with the environment, in order to minimize contamination of the compositions before and after the container is opened.

As is demonstrated in the Examples section that follows, the composition of the present invention can be efficiently used for transdermally administering testosterone. Such a transdermal delivery of testosterone or any other hormone evidently results in elevating the hormone serum level and can therefore be beneficial in the treatment of various conditions.

Thus, in a preferred embodiment of the present invention, the composition described hereinabove is packaged in a packaging material and is identified in print, in or on the package, for use in the treatment of a medical condition in which elevating a serum hormone level is beneficial. Examples of such medical conditions include, without limitation, hypogonadism, erectile dysfunction, hormone deficiency, AIDS wasting syndrome, breast cancer, postpartum breast pain or engorgement, reduced sex drive, depression, menopausal symptoms, energy loss, and loss of bone mass, as is detailed hereinbelow.

According to another aspect of the present invention, there is provided a method of transdermally delivering a hormone, such as testosterone, to the blood serum of a subject. The method is effected by providing an amount of a hydroalcoholic gel composition for topical application which comprises the hormone, a quaternary ammonium compound, an alcohol and a gelling agent, as described hereinabove, and contacting an amount of the composition with one or more biological surface(s) of the subject, to thereby deliver the hormone to the blood serum of the subject through the biological surface(s). The method according to this aspect of the present invention enables the use of a minimal amount of the applied composition and a minimal skin surface area onto which the composition is applied, while still achieving a desired testosterone level in a receiving medium (e.g., serum).

As can be seen by comparison of Examples 1 and 2, which compare the transdermal absorption through human skin of aqueous alcoholic gels comprising TWEEN-20 with a hydroalcoholic gel containing 0.1 weight percentage benzalkonium chloride, the amount of testosterone absorbed with benzalkonium chloride as penetration enhancer was significantly higher than with TWEEN-20. Specifically, the amount of testosterone in a receiver phase after 24 hours was found to be about 105 mcg with use of 0.1 weight percentage benzalkonium chloride while no difference was seen in formulations containing varying concentrations of Tween-20 as compared to those containing no penetration enhancer.

According to another aspect of the present invention, there is provided a method of treating a medical condition in which elevating a serum hormone level of a subject is beneficial. The method is effected by topically applying onto at least one biological surface of the subject, e.g. an inside arm, the back, the abdomen, a thigh, an armpit, a shoulder, or the scrotum, a pharmaceutically effective amount of the composition of the present invention as described herein.

The method according to this aspect of the present invention may be used to treat a medical condition in a human male, which includes hormone replacement therapy in males with a congenital or acquired deficiency or absence of endogenous testosterone (resulting in e.g hypogonadism, erectile dysfunction), and treatment of AIDS wasting syndrome in HIV infected men.

The hypogonadism may be primary hypogonadism, such as testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica); secondary hypogonadism, including Kaliman's Syndrome, Prader-Labhart-Willi's Syndrome, Laurence-Moon-Biedl's Syndrome, pituitary insufficiency/adenomas, Pasqualim's Syndrome, hemochromatosis, hyperprolactinemia, or pituitary-hypothalamic injury from tumors, trauma, radiation, or obesity; or age-related hypogonadism, resulting in physiological changes, including sexual dysfunction, decreased libido, loss of muscle mass, decreased bone density, depressed mood, and decreased cognitive function.

Symptoms of low testosterone include decreased sexual desire and ability (decreased libido), extreme tiredness, low energy, depression, and loss of certain male characteristics such as muscular build and deep voice.

The method of the present invention may be used to treat a medical condition in a human female which includes breast cancer and postpartum breast pain or engorgement, to enhance the sex drive, for relief of menopausal symptoms, restoration of lost energy, and to strengthen bone. Testosterone administration has also been found to be beneficial in young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

According to this aspect of the present invention, the hydroalcoholic gel composition of the present invention may be co-administered together with an additional pharmaceutically active ingredient suitable for treating the medical condition. As a non-limiting example, the composition may be used in conjunction with pharmaceuticals aimed at improving sexual performance or impotence, including agents to treat erectile dysfunction, such as VIAGRA,™, or increasing libido by increasing testosterone levels in men. These pharmaceuticals can be administered orally, intravenously or via any other route of administration. In another example, the composition may be used in conjunction with anti-depressants. In another example, non-drug therapies, such as, but not limited to, surgery, can be used in conjunction with the method according to this aspect of the present invention.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The phrase “topically applying” describes application onto one or more biological surface(s), by contacting a composition with the surface. Non-limiting examples of biological surfaces onto which the compositions of the present invention can be topically applied include one or more of the back, the abdomen, an inside arm, an armpit, a thigh, a shoulder, or the scrotum.

The composition is preferably applied to those regions of the skin which provide maximal systemic absorption of the hormonal active ingredient.

According to this aspect of the present invention, the composition of the present invention is preferably topically applied between two times a day and once in two days. More preferably, the composition is applied once a day, on a daily basis.

The phrase “phamaceutically effective amount” describes an amount of a composition that is sufficient to significantly induce a positive modification in the condition being treated, but low enough to avoid significant side effects, within the scope of sound judgment of the skilled artisan. Preferably, the amount of the applied composition is sufficient to elevate the blood serum level of the administered hormone from a subpotent concentration to a potent concentration, within about 24 hours after the administration. In the case of testosterone in a human male subject, the potent blood serum concentration ranges from about 300 ng/dl to about 1100 ng/dl.

A preferred amount of the composition of the present invention that upon application thereof results in the desired hormone level ranges between about 0.1 gram and 10 grams, preferably between about 1 gram and about 10 grams, more preferably between about 2 grams and about 10 grams, more preferably between about 3 grams and about 10 grams, more preferably between about 3 grams and about 10 grams, and more preferably between about 5 grams and about 10 grams. However, it should be noted that due to enhanced penetration induced by quaternary ammonium compounds, the amount of the composition of the present invention application thereof results in the desired hormone level can be less than 5 grams.

A representative example of a preferred composition according to an embodiment of the present invention, comprises about 1 weight percentage testosterone, and about 0.1 weight benzalkonium chloride, in a hydroalcoholic gel carrier comprising an alcohol and a gelling agent, which enables the use of a minimal amount of the applied composition and a minimal skin surface area onto which the composition is applied, while still achieving a desired testosterone level in a receiving medium (e.g., serum).

The present invention further encompasses processes for the preparation of the pharmaceutical compositions described above. These processes generally comprise admixing the active ingredients described hereinabove and the pharmaceutically acceptable carrier. In cases where other agents or active agents, as is detailed hereinabove, are present in the compositions, the process includes admixing these agents together with the active ingredients and the carrier. A variety of exemplary formulation techniques that are usable in the process of the present invention is described, for example, in Harry's Cosmeticology, Seventh Edition, Edited by J B Wilkinson and R J Moore, Longmann Scientific & Technical, 1982, Chapter 13 “The Manufacture of Cosmetics” pages 757-799.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

This example compares the transdermal absorption through human skin of testosterone from aqueous alcoholic gels containing 1.0% (w/w) testosterone, 0.0%, 0.1%, 0.7% or 2.0% of Tween-20 and 69.0% of ethanol. Carbopol 940 is used as the gelling agent in the gel formulations. The test compositions are applied to provide about 55 milligrams (mg) of the composition per square centimeter (cm²) of human skin.

The tests are run in standard diffusion cells with ethanol-water mixture (50:50) as the receptor fluid (surface area 1.77 cm², temperature 37 degree Celsius). The following Table 1 shows the concentration of the enhancer (Tween-20) in the formulations and the total amount of testosterone present in receiver phase after 24 hours for each formulation.

Each test was run for 24 hours under non-occluded conditions with the finite dose of the test formulation.

TABLE 1

| | Total amount of | Percentage of applied |
|---|---|---|
| Concentration | testosterone in receiver | testosterone that reached |
| of Tween 20 in | phase after 24 hours | receiver phase after 24 |
| formulation (% w/w) | (microgram) | hours (%) |
| 0.0 | 39.5 | 3.95 |
| 0.1 | 29.1 | 2.91 |
| 0.7 | 38.3 | 3.83 |
| 2.0 | 40.35 | 4.035 |

### Example 2

This example compares the transdermal absorption through human skin of testosterone from aqueous alcoholic gels containing 1.0% (w/w) testosterone, 0.0% or 0.1% of benzalkoniun chloride and 69.0% of ethanol. Carbopol 940 is used as the gelling agent in the gel formulations. The test compositions are applied to provide about 55 milligrams (mg) of the composition per square centimeter (cm²) of human skin.

The tests are run in standard diffusion cells with ethanol-water mixture (50:50) as the receptor fluid (surface arca 1.77 cm², temperature 37 degree Celsius). The following Table 2 shows the concentration of the enhancer (benzalkonium chloride) in the formulations and the total amount of testosterone present in receiver phase after 24 hours for each formulation.

Each test was run for 24 hours under non-occluded conditions with the finite dose of the test formulation.

TABLE 2

| Concentration | Total amount of | Percentage of applied |
|---|---|---|
| of Benzalkonium | testosterone in receiver | testosterone that reached |
| chloride in the | phase after 24 hours | receiver phase after 24 |
| formulation (% w/w) | (microgram) | hours (%) |
| 0.0 | 39.5 | 3.95 |
| 0.01 | 58.8 | 5.88 |
| 0.02 | 70.5 | 7.05 |
| 0.1 | 97.3 | 9.73 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described with reference to specific embodiments thereof, many alternatives, modifications and variations will be apparent to those skilled in the art. Accordingly, it is intended that the present invention embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

1. A hydroalcoholic gel pharmaceutical composition for topical application comprising a pharmaceutically active ingredient, a penetration enhancer, an alcohol and a gelling agent, wherein said pharmaceutically active ingredient is a hormone, and said penetration enhancer is a quaternary ammonium compound.

2. The hydroalcoholic gel pharmaceutical composition of claim 1, being capable, upon application of an amount of the composition onto at least one biological surface of a subject, of elevating a blood serum concentration of said hormone in said subject from a subpotent concentration to a potent concentration within about 24 hours after said application.

3. The hydroalcoholic gel pharmaceutical composition of claim 2, wherein said amount ranges between about 0.1 grams and about 10 grams.

4. The hydroalcoholic gel pharmaceutical composition of claim 2, wherein said amount ranges between about 3 milligrams and about 100 milligrams per square centimeter of said at least one biological surface.

5. The hydroalcoholic gel pharmaceutical composition of claim 4, wherein said amount ranges between about 4 milligrams and about 60 milligrams per square centimeter of said at least one biological surface.

6. The hydroalcoholic gel pharmaceutical composition of claim 1, wherein a concentration of said quaternary ammonium compound ranges between about 0.04 weight percentages and about 0.4 weight percentages.

7. The hydroalcoholic gel pharmaceutical composition of claim 6, wherein a concentration of said quaternary ammonium compound ranges between about 0.07 weight percentage and about 0.1 weight percentage.

8. The hydroalcoholic gel pharmaceutical composition of claim 1, wherein said quaternary ammonium compound is selected from the group consisting of benzalkonium chloride, benzethonium chloride, hexadecyltrethylammonium bromide, methylbenzethonium chloride, cetalkonium chloride, centrimonium bromide, domiphen bromide, domiphen chloride, domiphen fluoride, cetyl pyridinium chloride, dequalinium chloride, the cis isomer of 1-(3-chloroallyl)-3,5,7-triaza-1-azoniaadamantane chloride, cetyl trimethylammonium bromide, benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; N,N-(diethyl-N-[2-[4-(1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride.

9. The hydroalcoholic gel pharmaceutical composition of claim 8, wherein said quaternary ammonium compound is benzalkonium chloride.

10. The hydroalcoholic gel pharmaceutical composition of claim 1, wherein said hormone is selected from the group consisting of an androgenic hormone, an estrogenic hormone and a progestogenic hormone.

11. The hydroalcoholic gel pharmaceutical composition of claim 10, wherein said hormone is selected from the group consisting of methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydrocpiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone, estrone, estradiol and estriol, progesterone, pharmaceutically acceptable esters thereof, salts thereof, and combinations of any of the foregoing.

12. The hydroalcoholic gel pharmaceutical composition of claim 11, wherein said hormone is testosterone.

13. The hydroalcoholic gel pharmaceutical composition of claim 1, wherein a concentration of said hormone ranges between about 0.5 weight percentages and about 5 weight percentages.

14. The hydroalcoholic gel pharmaceutical composition of claim 13, wherein a concentration of said hormone is about 1 weight percentage.

15. The hydroalcoholic gel pharmaceutical composition of claim 1, wherein said alcohol is a C2-C4 alcohol.

16. The hydroalcoholic gel pharmaceutical composition of claim 15, wherein said C2-C4 alcohol is selected from the group comprising ethanol and isopropanol.

17. The hydroalcoholic gel pharmaceutical composition of claim 16, wherein said C2-C4 alcohol is ethanol.

18. The hydroalcoholic gel pharmaceutical composition of claim 15, wherein a concentration of said C2-C4 alcohol ranges between about 40 weight percentages and about 90 weight percentages.

19. The hydroalcoholic gel pharmaceutical composition of claim 18, wherein a concentration of said C2-C4 alcohol ranges between about 55 weight percentages and about 70 weight percentages.

20. The hydroalcoholic gel pharmaceutical composition of claim 19, wherein a concentration of said C2-C4 alcohol is about 69 weight percentages.

21. The hydroalcoholic gel pharmaceutical composition of claim 1, wherein said gelling agent is selected from the group consisting of a polymeric thickening agent, a fatty alcohol, a fatty acid, and a fatty acid alkali salt, an inorganic gelling agent and any mixture thereof.

22. The hydroalcoholic gel pharmaceutical composition of claim 1, wherein said gelling agent comprises a polyacrylic acid.

23. The hydroalcoholic gel pharmaceutical composition of claim 21, wherein said polymeric thickening agent comprises a cellulosic ether.

24. The hydroalcoholic gel pharmaceutical composition of claim 23, wherein said cellulosic ether is selected from the group consisting of carboxymethylcellulose, hydroxypropyl cellulose and hydroxyethylcellulose.

25. The hydroalcoholic gel pharmaceutical composition of claim 21, wherein said polymeric thickening agent is selected from the group consisting of xanthan gum and guar gum.

26. The hydroalcoholic gel pharmaceutical composition of claim 1, wherein a concentration of said gelling agent ranges between about 0.1 weight percentage and about 5 weight percentages.

27. The hydroalcoholic gel pharmaceutical composition of claim 26, wherein a concentration of said gelling agent ranges between about 0.1 weight percentage and about 2 weight percentages.

28. The hydroalcoholic gel pharmaceutical composition of claim 1, further comprising a penetration co-enhancer.

29. The hydroalcoholic gel pharmaceutical composition of claim 28, wherein said penetration co-enhancer is a glycol.

30. The hydroalcoholic gel pharmaceutical composition of claim 1, further comprising an additional pharmaceutically active ingredient.

31. The hydroalcoholic gel pharmaceutical composition of claim 1, further comprising at least one additive.

32. The hydroalcoholic gel pharmaceutical composition of claim 31, wherein said at least one additive is selected from the group consisting of a moisturizing agent and an emollient.

33. The hydroalcoholic gel pharmaceutical composition of claim 31, wherein a concentration of said at least one additive ranges between about 1 weight percentage and about 5 weight percentages.

34. The hydroalcoholic gel pharmaceutical composition of claim 31, wherein said at least one additive comprises glycerin.

35. The hydroalcoholic gel pharmaceutical composition of claim 32, wherein said emollient is selected from the group comprising dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

36. The hydroalcoholic gel pharmaceutical composition of claim 31, wherein said at least one additive is selected from the group consisting of a humectant, a deodorant agent, an antiperspirant, a stabilizing agent, a pH adjusting agent, a preservative, an emulsifier, an occlusive agent, a solubilizing agent, a colorant, and a surfactant.

37. The hydroalcoholic gel pharmaceutical composition of claim 1, packaged in a packaging material and identified in print, in or on said packaging material, for use in the treatment of a medical condition in which elevating a serumn hormone level in a subject is beneficial.

38. The hydroalcoholic gel pharmaceutical composition of claim 37, wherein said subject is a human male.

39. The hydroalcoholic gel pharmaceutical composition of claim 38, wherein said medical condition is selected from the group consisting of primary hypogonadism, secondary hypogonadism, age-related hypogonadism, hormone deficiency, erectile dysfunction, AIDS wasting syndrome, reduced sex drive, energy loss, loss of bone mass, extreme tiredness, low energy, and depression.

40. The hydroalcoholic gel pharmaceutical composition of claim 37, wherein said subject is a human female.

41. The hydroalcoholic gel pharmaceutical composition of claim 40, wherein said medical condition is selected from the group consisting of breast cancer, postpartum breast pain or engorgement, reduced sex drive, menopausal symptoms, energy loss, loss of bone mass, extreme tiredness, low energy, and depression.

42. The hydroalcoholic gel pharmaceutical composition of claim 40, wherein said human female is selected from the group consisting of young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

43. A hydroalcoholic gel pharmaceutical composition for topical application comprising testosterone, benzalkonium chloride, a C2-C4 alcohol and a gelling agent.

44. The hydroalcoholic gel pharmaceutical composition of claim 43, being capable, upon application of an amount of the composition onto at least one biological surface of a subject, of elevating a blood serum concentration of said testosterone in said subject from a subpotent concentration to a potent concentration within about 24 hours after said application.

45. The hydroalcoholic gel pharmaceutical composition of claim 44, wherein said amount ranges between about 0.1 grams and about 10 grams.

46. The hydroalcoholic gel pharmaceutical composition of claim 43, wherein a concentration of said benzalkonium chloride ranges between about 0.04 weight percentages and about 0.4 weight percentages.

47. The hydroalcoholic gel pharmaceutical composition of claim 46, wherein a concentration of said benzalkonium chloride ranges between about 0.07 weight percentage and about 0.1 weight percentages.

48. The hydroalcoholic gel pharmaceutical composition of claim 43, wherein a concentration of said testosterone ranges between about 0.5 and about 5 weight percentages.

49. The hydroalcoholic gel pharmaceutical composition of claim 48, wherein a concentration of said testosterone is about 1 weight percentage.

50. The hydroalcoholic gel pharmaceutical composition of claim 43, wherein said C2-C4 alcohol is selected from the group comprising ethanol and isopropanol.

51. The hydroalcoholic gel pharmaceutical composition of claim 50, wherein said C2-C4 alcohol is ethanol.

52. The hydroalcoholic gel pharmaceutical composition of claim 43, wherein a concentration of said C2-C4 alcohol ranges between about 40 weight percentages and about 90 weight percentages.

53. The hydroalcoholic gel pharmaceutical composition of claim 52, wherein a concentration of said C2-C4 alcohol ranges between about 55 weight percentages and about 70 weight percentages.

54. The hydroalcoholic gel pharmaceutical composition of claim 53, wherein a concentration of said C2-C4 alcohol is about 69 weight percentages.

55. The hydroalcoholic gel pharmaceutical composition of claim 43, wherein said gelling agent is selected from the group consisting of a polymeric thickening agent, a fatty alcohol, a fatty acid, and a fatty acid alkali salt, an inorganic gelling agent and any mixture thereof.

56. The hydroalcoholic gel pharmaceutical composition of claim 43, wherein said gelling agent comprises a polyacrylic acid.

57. The hydroalcoholic gel pharmaceutical composition of claim 43, further comprising an additional pharmaceutically active ingredient.

58. The hydroalcoholic gel pharmaceutical composition of claim 43, packaged in a packaging material and identified in print, in or on said packaging material, for use in the treatment of a medical condition in which elevating a serum hormone level in a subject is beneficial.

59. The hydroalcoholic gel pharmaceutical composition of claim 58, wherein said subject is a human male.

60. The hydroalcoholic gel pharmaceutical composition of claim 59, being capable, upon application of an amount of the composition onto at least one biological surface of said male subject, of elevating a blood serum concentration of said testosterone in said human male to a value ranging between about 300 ng/dl and about 1100 ng/dl, within 24 hours after said application.

61. The hydroalcoholic gel pharmaceutical composition of claim 59, wherein said medical condition is selected from the group consisting of primary hypogonadism, secondary hypogonadism, age-related hypogonadism, hormone deficiency, erectile dysfunction, AIDS wasting syndrome, reduced sex drive, energy loss, loss of bone mass,extreme tiredness, low energy, and depression.

62. The hydroalcoholic gel pharmaceutical composition of claim 58, wherein said subject is a human female.

63. The hydroalcoholic gel pharmaceutical composition of claim 62, wherein said medical condition is selected from the group consisting of breast cancer, postpartum breast pain or engorgement, reduced sex drive, menopausal symptoms, energy loss, loss of bone mass, extreme tiredness, low energy, and depression.

64. The hydroalcoholic gel pharmaceutical composition of claim 62, wherein said human female is selected from the group consisting of young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

65. A method of transdermally delivering a hormone to the blood serum of a subject, the method comprising:
providing a hydroalcoholic gel pharmaceutical composition for topical application including said hormone, a quaternary ammonium compound, an alcohol and a gelling agent; and
contacting an amount of said topical pharmaceutical composition with at least one biological surface of said subject, to thereby deliver said hormone to said blood serum through said biological surface.

66. The method of claim 65, wherein said amount of said pharmaceutical hydroalcoholic gel composition ranges between about 0.1 gram and about 10 grams.

67. The method of claim 65, wherein said amount of said hydroalcoholic gel pharmaceutical composition ranges between about 3 milligrams and about 100 milligrams per square centimeter of said at least one biological surface.

68. The method of claim 67, wherein said amount ranges between about 4 milligrams and about 60 milligrams per square centimeter of said at least one biological surface.

69. The method of claim 65, wherein a concentration of said hormone in said blood serum of said subject is elevated from a subpotent concentration to a potent concentration within about 24 hours after said contacting.

70. The method of claim 65, wherein said at least one biological surface is selected from the group consisting of the abdomen, an armpit, an inside arm, the back, a thigh, a shoulder, and the scrotum.

71. The method of claim 65, wherein a concentration of said quaternary ammonium compound ranges between about 0.04 weight percentages and about 0.4 weight percentages.

72. The method of claim 71, wherein a concentration of said quaternary ammonium compound ranges between about 0.07 weight percentages and about 0.1 weight percentages.

73. The method of claim 65, wherein said quaternary ammonium compound is selected from the group consisting of benzalkonium chloride, benzethonium chloride, cetrimide, and methylbenzethonium chloride hexadecyltrethylammonium bromide, methylbenzethonium chloride, cetalkonium chloride, centrimonium bromide, domiphen bromide, domiphen chloride, domiphen fluoride, cetyl pyridinium chloride, dequalinium chloride, the cis isomer of 1-(3-chloroallyl)-3,5,7-triaza-1-azoniaadamantane chloride, cetyl trimethylammonium bromide, benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; N,N-(diethyl-N-[2-[4-(1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride.

74. The method of claim 73, wherein said quaternary ammonium compound is benzalkonium chloride.

75. The method of claim 65, wherein said hormone is selected from the group consisting of an androgenic hormone, an estrogenic hormone and a progestogenic hormone.

76. The method of claim 75, wherein said hormone is selected from the group consisting of methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progtesterone acetate, 3-ketodesogestrel, megestrol, melengestrol acetate, norethisterone, estrone, estradiol and estriol, progesterone, pharmaceutically acceptable esters, salts thereof, and combinations of any of the foregoing.

77. The method of claim 76, wherein said hormone is testosterone.

78. The method of claim 65, wherein a concentration of said hormone ranges between about 0.5 weight percentages and about 5 weight percentages of the total weight of said composition.

79. The method of claim 78, wherein a concentration of said hormone is about 1 weight percentage of the total weight of said composition.

80. The method of claim 65, wherein said alcohol is a C2-C4 alcohol.

81. The method of claim 80, wherein said C2-C4 alcohol is selected from the group comprising ethanol and isopropanol.

82. The method of claim 81, wherein said C2-C4 alcohol is ethanol.

83. The method of claim 80, wherein a concentration of said C2-C4 alcohol ranges between about 40 weight percentages and about 90 weight percentages of the total weight of said composition.

84. The method of claim 83, wherein a concentration of said C2-C4 alcohol ranges between about 55 weight percentages and about 70 weight percentages of the total weight of said composition.

85. The method of claim 84, wherein a concentration of said C2-C4 alcohol is about 69 weight percentages of the total weight of said composition.

86. The method of claim 65, wherein said gelling agent is selected from the group consisting of a polymeric thickening agent, a fatty alcohol, a fatty acid, and a fatty acid alkali salt, an inorganic gelling agent and any mixture thereof.

87. The method of claim 65, wherein said gelling agent comprises a polyacrylic acid.

88. The method of claim 86, wherein said polymeric thickening agent comprises a cellulosic ether.

89. The method of claim 88, wherein said cellulosic ether is selected from the group consisting of carboxymethylcellulose, hydroxypropyl cellulose and hydroxyethylcellulose.

90. The method of claim 86, wherein said polymeric thickening agent is selected from the group consisting of xanthan gum and guar gum.

91. The method of claim 65, wherein a concentration of said gelling agent ranges between about 0.1 weight percentage and about 5 weight percentages of the total weight of said composition.

92. The method of claim 91, wherein a concentration of said gelling agent ranges between about 0.1 weight percentage and about 2 weight percentages of the total weight of said composition.

93. The method of claim 65, wherein said pharmaceutical composition further comprises a penetration co-enhancer.

94. The method of claim 93, wherein said penetration co-enhancer is a glycol.

95. The method of claim 65, wherein said pharmaceutical composition further comprises an additional pharmaceutically active ingredient.

96. The method of claim 65, wherein said pharmaceutical composition further comprises at least one additive.

97. The method of claim 96, wherein said at least one additive is selected from the group consisting of a moisturizing agent and an emollient.

98. The method of claim 96, wherein a concentration of said at least one additive ranges between about 1 weight percentage and about 5 weight percentages of the total weight of said composition.

99. The method of claim 96, wherein said at least one additive comprises glycerin.

100. The method of claim 97, wherein said emollient is selected from the group comprising dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

101. The method of claim 96, wherein said at least one additive is selected from the group consisting of a humectant, a deodorant agent, all antiperspirant, a stabilizing agent, a pH adjusting agent, a preservative, an emulsifier, an occlusive agent, a solubilizing agent, a colorant, and a surfactant.

102. A method of treating a medical condition in which elevating a blood serum hormone level in a subject is beneficial, the method comprising:
providing a hydroalcoholic gel pharmaceutical composition for topical application including said hormone, a quaternary ammonium compound, an alcohol and a gelling agent;
topically applying onto at least one biological surface of said subject a pharmaceutically effective amount of said topical pharmaceutical composition, thereby elevating said blood serum hormone level in said subject and treating said medical condition.

103. The method of claim 102, wherein said pharmaceutically effective amount of said hydroalcoholic gel pharmaceutical composition ranges between about 0.1 gram and about 10 grams.

104. The method of claim 102, wherein said amount of said hydroalcoholic gel pharmaceutical composition ranges betweeen about 3 milligrams and about 100 milligrams per square centimeter of said at least one biological surface.

105. The method of claim 104, wherein said amount of said hydroalcoholic gel pharmaceutical composition ranges between about 4 milligrams and about 60 milligrams per square centimeter of said at least one biological surface.

106. The method of claim 102, wherein said hormone level is elevated from a subpotent concentration to a potent concentration within about 24 hours after said topical application.

107. The method of claim 102, wherein said at least one biological surface is selected from the group consisting of the abdomen, an armpit, an inside arm, the back, a thigh, a shoulder, and the scrotum.

108. The method of claim 102, wherein a concentration of said quaternary ammonium compound ranges between about 0.04 weight percentages and about 0.4 weight percentages.

109. The method of claim 108, wherein a concentration of said quaternary ammonium compound ranges between about 0.07 weight percentages and about 0.1 weight percentages.

110. The method of claim 102, wherein said quaternary ammonium compound is selected from the group consisting of benzalkonium chloride, benzethonium chloride, cetrimide, methylbenzethonium chloride, hexadecyltrethylammonium bromide, methylbenzethonium chloride, cetalkonium chloride, centrimonium bromide, domiphen bromide, domiphen chloride, domiphen fluoride, cetyl pyridinium chloride, dequalinium chloride, the cis isomer of 1-(3-chloroallyl)-3,5,7-triaza-1-azoniaadamantane chloride, cetyl trimethylammonium bromide, benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; N,N-(dietyl-N-[2-[4-(1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride.

111. The method of claim 110, wherein said quaternary ammonium compound is benzalkonium chloride.

112. The method of claim 102, wherein said hormone is selected from the group consisting of an androgenic hormone, an estrogenic hormone and a progestogenic hormone.

113. The method of claim 112, wherein said hormone is selected from the group consisting of methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20α-diol sulfate, 5α-pregnan-3β-ol-20-one, 16,5α-pregnen-3β-ol-20one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone, estrone, estradiol and estriol, progesterone, pharmaceutically acceptable esters thereof, salts thereof, and combinations of any of the foregoing.

114. The method of claim 113, wherein said hormone is testosterone.

115. The method of claim 102, wherein a concentration of said hormone ranges between about 0.5 weight percentages and about 5 weight percentages of the total weigh of the said composition.

116. The method of claim 115, wherein a concentration of said hormone is about 1 weight percentage of the total weight of the said composition.

117. The method of claim 102, wherein said alcohol is a C2-C4 alcohol.

118. The method of claim 117, wherein said C2-C4 alcohol is selected from the group comprising ethanol and isopropanol.

119. The method of claim 118, wherein said C2-C4 alcohol is ethanol.

120. The method of claim 117, wherein a concentration of said C2-C4 alcohol ranges between about 40 weight percentages and about 90 weight percentages of the total weight of the said composition.

121. The method of claim 120, wherein a concentration of said C2-C4 alcohol ranges between about 55 weight percentages and about 70 weight percentages of the total weight of the said composition.

122. The method of claim 121, wherein a concentration of said C2-C4 alcohol is about 69 weight percentages of the total weight of the said composition.

123. The method of claim 102, wherein said gelling agent is selected from the group consisting of a polymeric thickening agent, a fatty alcohol, a fatty acid, and a fatty acid alkali salt, an inorganic gelling agent and any mixture thereof.

124. The method of claim 102, wherein said gelling agent comprises a polyacrylic acid.

125. The method of claim 123, wherein said polymeric thickening agent comprises a cellulosic ether.

126. The method of claim 125, wherein said cellulosic ether is selected from the group consisting of carboxymethylcellulose, hydroxypropyl cellulose and hydroxyethylcellulose.

127. The method of claim 123, wherein said polymeric thickening agent is selected from the group consisting of xanthan gum and guar gum.

128. The method of claim 85, wherein a concentration of said gelling agent ranges between about 0.1 weight percentage and about 5 weight percentages of the total weight of the said composition.

129. The method of claim 102, wherein a concentration of said gelling agent ranges between about 0.1 weight percentage and about 2 weight percentages of the total weight of the said composition.

130. The method of claim 102, wherein said pharmaceutical composition further comprises a penetration co-enhancer.

131. The method of claim 130, wherein said penetration co-enhancer is a glycol.

132. The method of claim 102, wherein said pharmaceutical composition further comprises an additional pharmaceutically active ingredient.

133. The method of claim 102, wherein said pharmaceutical composition further comprises at least one additive.

134. The method of claim 133, wherein said at least one additive is selected from the group consisting of a moisturizing agent and an emollient.

135. The method of claim 133, wherein a concentration of said at least one additive ranges between about 1.0 weight percentages and about 5 weight percentages of the total weight of the said composition.

136. The method of claim 133, wherein said at least one additive comprises glycerin.

137. The method of claim 134, wherein said emollient is selected from the group comprising dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

138. The method of claim 133, wherein said at least one additive is selected from the group consisting of a humectant, a deodorant agent an antiperspirant, a stabilizing agent, a stabilizing agent, a pH adjusting agent, a preservative, an emulsifier, an occlusive agent, a solubilizing agent, a colorant, and a surfactant.

139. The method of claim 102, wherein said subject is a human male.

140. The method of claim 139, wherein said medical condition is selected from the group consisting of primary hypogonadism, secondary hypogonadism, age-related hypogonadism, hormone deficiency, erectile dysfunction, AIDS wasting syndrome, reduced sex drive, energy loss, loss of bone mass, extreme tiredness, low energy, and depression.

141. The method of claim 102, wherein said subject is a human female.

142. The method of claim 141, wherein said medical condition is selected from the group consisting of breast cancer, postpartum breast pain or engorgement, reduced sex drive, menopausal symptoms, energy loss, loss of bone mass, extreme tiredness, low energy, and depression.

143. The method of claim 141, wherein said human female is selected from the group consisting of young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

144. The method of claim 102, further comprising co-administering to said subject an additional pharmaceutically active ingredient suitable for treating said medical condition.
